(19) United States
(12) Patent Application Publication
(10) Pub. No.: US 2017/0343523 A1
(43) Pub. Date: Nov. 30, 2017

(54) ANALYSIS SYSTEM FOR ANALYZING WATER AND WASTEWATER

(71) Applicant: LAR PROCESS ANALYSERS AG, Berlin (DE)

(72) Inventors: Werner Arts, Berlin (DE); Wolfgang Genthe, Berlin (DE)

(73) Assignee: LAR PROCESS ANALYSERS AG, Berlin (DE)

(21) Appl. No.: 15/534,275

(22) PCT Filed: Dec. 8, 2015
(86) PCT No.: PCT/DE2015/100519

§ 371 Date: Jun. 8, 2017

(30) Foreign Application Priority Data
Dec. 8, 2014 (DE) 10 2014 118 138.7

(51) Int. Cl. G01N 33/18; B01L 3/00; G01N 1/14; G01N 31/12

(52) U.S. Cl. CPC G01N 33/1806; G01N 31/12; B01L 3/50; G01N 1/14; B01L 2200/16; B01L 2200/0642; B01L 2200/025; B01L 2300/0672; B01L 2300/14; B01L 2400/0478; B01L 2300/0832

(57) ABSTRACT

Disclosed is an analysis system for analyzing water and wastewater, comprising an analysis device that includes a device housing which accommodates device components and which has an inlet on a housing surface, said inlet being designed as an injection port through which a substance to be analyzed can be introduced into a device component when the device housing is closed, and comprising a syringe that includes an injection needle outlet, the surface normal of which is congruent with the longitudinal axis; and/or the syringe includes an automatic ejection element for ejecting a predetermined amount of substance within a predetermined injection period.

### BACKGROUND

The invention relates to an analysis system for analyzing water and waste water, comprising an analysis device including a device housing and an injection port for introducing a sample into the device, as well as a syringe.

Such analysis systems are known in principle and are manufactured and offered by the applicant.

### SUMMARY

The invention is based on the object of providing an improved analysis system of this type, which, in particular, can provide reliably accurate and exactly reproducible analysis results.

This object is achieved by an analysis device having one or more features according to the invention. Advantageous further developments of the invention are described below and in the claims.

First of all, the invention involves the idea of modifying the associated syringe, deviating from conventional syringes, in the interest of performing the injection process optimally and reproducibly. According to a first modification, an outlet opening of the needle is provided, the surface normal of which coincides with (or is at least parallel to) the needle's longitudinal axis. According to a second modification, an automatically operating ejection element for ejecting a predetermined amount of substance in a predetermined injection period is provided. Advantageously, the syringe of the proposed systems has both features in combination.

In one embodiment of the invention, the injection port is connected to a cylindrical reaction vessel for thermal disintegration of the substance to be analyzed, and it has guiding means for guiding the syringe into a predetermined injection position. Specifically, the guiding means are, in accordance with the shape of the injection needle, formed such that the longitudinal axis of the inserted injection needle coincides with the longitudinal axis of the reaction vessel. In particular, the guiding means comprise a cylindrical or conical guide sleeve. In a further embodiment, which may be combined with the above-mentioned embodiment, the guiding means includes a stopper for limiting the depth of penetration of the injection needle into the reaction vessel.

In a further embodiment of the invention the ejection element is formed as a lockable compression spring, which acts on the plunger of the syringe. The compression spring, which can be configured, for example, as a steel cylinder spring, thus replaces a manual operation of the syringe. Unlike during manual operation, the predetermined spring characteristic of the compression spring ensures a precisely reproducible sample output per unit of time. However, this important effect would not necessarily need to be implemented by a compression spring (in a particularly simple and inexpensive embodiment), but can, for example, also be implemented by a small linear motor or a hydraulic or pneumatic drive with predetermined a predetermined output characteristic.

According to the second aspect, the invention involves the idea that a carrier gas supply is connected to the reaction vessel on the input side, the carrier gas supply including controllable means for setting the pressure and flow rate of a carrier gas to be supplied to the reaction vessel. In addition to a sample supply which involves a precisely dimensioned amount and for which the location and the direction of the ejection of the sample into the reaction vessel is precisely predetermined, also an accurate and reproducible control of the carrier gas flow for transporting the sample to a detection device is of great importance for accurate and reproducible analysis results. It has been found that a simple flow restriction, such as by means of a reducing valve, is not sufficient for the accuracy requirements of an analysis system suitable for laboratory use.

In particular, provision is made so that the carrier gas supply comprises a first branch (partial flow) for supplying nitrogen and a second branch (partial flow) for supplying oxygen, wherein, in the first branch, a first pressure regulator and flow regulator for pressure regulation or flow regulation of supplied nitrogen and, in the second branch, a second pressure regulator and flow regulator for pressure regulation or flow regulation of oxygen to be mixed with the nitrogen are provided. Here, the first and second pressure and flow regulators are, in particular, configured and adapted such that a dilution by a factor of at least 10, particularly at least 100, and more particularly of about 1000, can be achieved during the mixing of the oxygen with the nitrogen. It is understood that precision flow regulators should be used for realizing such low dilutions with sufficient accuracy.

According to the aforementioned third aspect, the invention is based on the idea of allowing the actual evaluation mode to be post-processed or influenced depending on certain features of the analytical result. The inventors have found that, in the determination of relevant components of water or wastewater samples, the actual sample composition, as well as peculiarities of the analysis process, may significantly affect the signal shape of a time-dependent detection signal, and that the advantage of “manually” influencing the evaluation process results therefrom.

Since, in particular, the temporal signal characteristics are very different for oxygen, carbon dioxide, nitrogen or phosphorus concentrations in the water or waste water measured as a function of time, partially useful and comparable analysis results may only be obtained with a fixed integration time. Therefore, preferably, provision is made so that the post-processing device for manually readjusting an integration time for integrating the respective time-dependent concentration values detected is formed.

### BRIEF DESCRIPTION OF THE DRAWINGS

Additional features and advantages of the invention are also apparent from the following brief description of an example embodiment with reference to the figures. In the figures:

FIG. 1 is a schematic diagram of essential device components of an analysis device according to an embodiment,

FIG. 2 is a perspective view of that analysis device,

FIG. 3 is a schematic longitudinal sectional view of a syringe having an injection needle inserted into the injection port according to an example embodiment of the analysis system, and

FIGS. 4A and 4B are diagrams for illustrating an aspect of the invention.

### DETAILED DESCRIPTION OF THE PREFERRED EMBODIMENTS

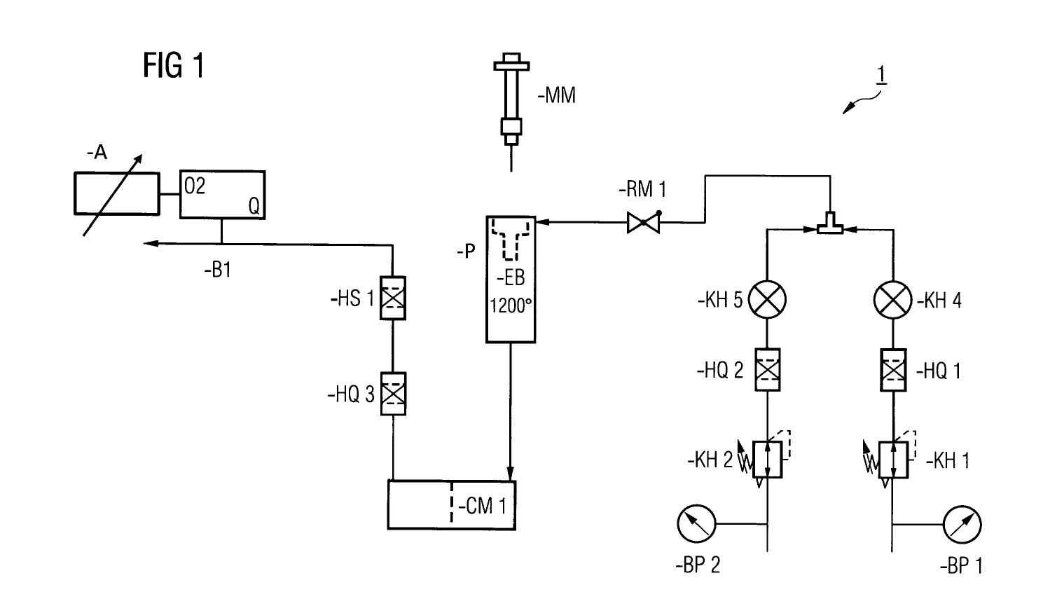

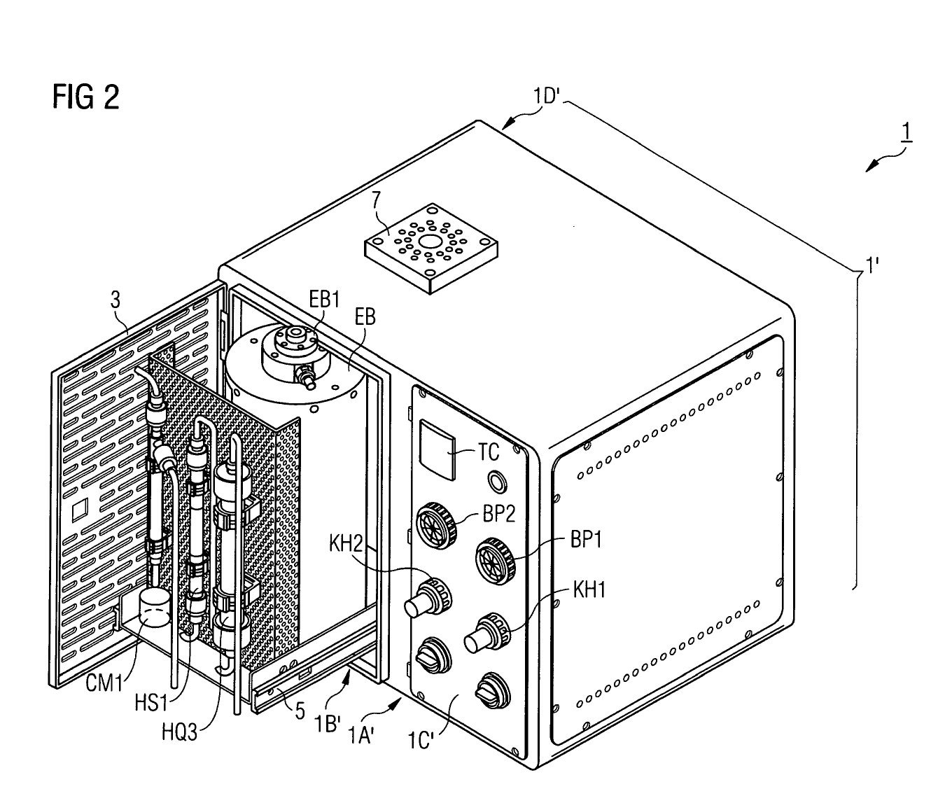

FIGS. 1 and 2 schematically show essential parts of an analysis device 1 for determining the chemical oxygen demand (CSB or COD) of water or waste water. The basic structure of such devices and the functionality thereof are known in the art and are therefore not described here, since they are irrelevant for the understanding of the present invention.

The analysis device 1 comprises a thermal reaction vessel or a furnace EB, into which a water sample can be injected using a syringe MM via an injection port P arranged on the furnace top and in which the sample is thermally disintegrated. The furnace is supplied with a carrier gas flow which is composed of air and nitrogen via a check valve RM 1. The carrier gas flow is controlled by an air pressure regulator KH 1, a nitrogen pressure regulator KH2, an air flow regulator KH4 and a nitrogen flow regulator KH5 and filtered, on the input side of the furnace, by means of a first and second fine filter HQ1, HQ2. On the input side of the furnace, also an air-pressure indicator BP1 and a nitrogen pressure indicator BP2 are provided.

On the output side of the furnace, the gas flow first arrives at a condensate vessel CM 1, and the non-condensed portion is then passed through a quartz wool filter HQ3 and an acid trap HS1 before it reaches the oxygen detector B1, which eventually outputs a (electric) measurement to an adjustable evaluation device A, at which, in particular, an integration time for integrating a oxygen detection signal detected as a function of time is provided; see below for further details.

FIG. 2 shows the analysis device 1 in a perspective view in a state in which the device housing 1′ is partially open and in which part of the device components is withdrawn from the housing. The device housing has substantially the shape of a square prism, and in the device front 1A′ an opening 1B′ is provided which can be closed by a perforated door 3 hinged at the left edge of the device front.

A carriage 5 having dimensions adapted to the opening 1B′, on which the furnace EB, the condensate vessel CM 1, the quartz wool filter HQ3 and the acid trap HS1 are arranged, can be pulled out of the case to such an extent that said components are freely accessible. In the retracted state of the carriage 5, the device housing 1′ is closed by the door 3.

On the right side of the front panel 1A′ a panel IC is located, on which a plurality of operating and display elements are arranged, including a temperature indicator/control TC and the setting regulators KH1 and KH2 for air or nitrogen pressure and the associated display elements BP1 and BP1.

On top of the device 1D′ the injection port P is located, the structure and dimensions of which are adapted to those of the syringe MM shown in FIG. 1 and which communicates to a corresponding injector valve EB1 of the furnace EB within the device when the furnace is in its normal operating position within the device housing.

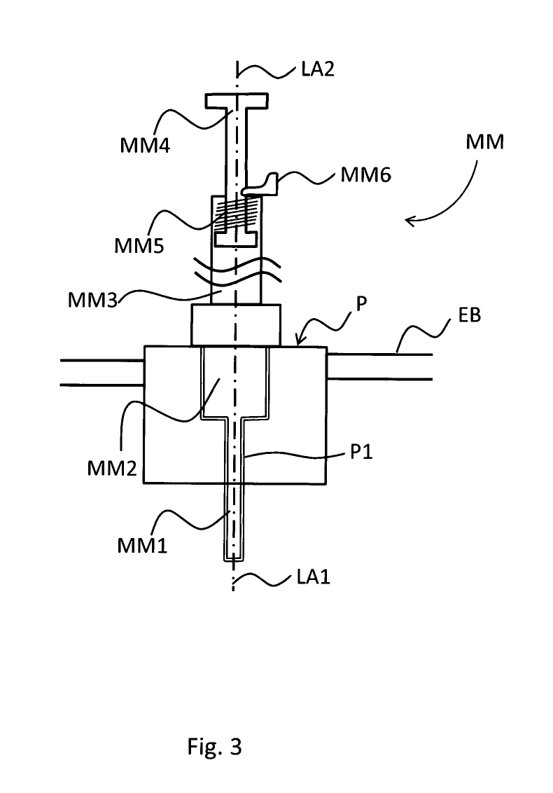

FIG. 3 shows, in a longitudinal sectional view, a sketch of the basic structure and the concerted geometrical configuration of the syringe MM and the injection port P of the furnace EB of the analysis system. The injection port P comprises, over its longitudinal course, an essentially cylindrically shaped guide sleeve P1, the diameter and length of which are adjusted to the corresponding dimensions of an injection needle MM 1 of the syringe MM and the longitudinal axis of which coincides with a longitudinal axis LA1 of the furnace EB which is shaped cylindrically in its basic form. At the top of the injection port P a bore P2 with an enlarged diameter is provided, the dimensions of which are adapted to those of a needle hub MM2 of the syringe and the lower end face of which acts as a stopper for depth limitation during insertion of the syringe. This stopper ensures an exactly predetermined position of the needle end, which is cut off at a right angle to the needle longitudinal axis LA2 of the syringe, in the furnace EB, and thus an exactly predetermined point of injection.

In the syringe reservoir MM3, a syringe plunger MM4 is supported in a longitudinally displaceable manner, the free end of which is configured in a conventional manner for manually withdrawing a sample. At the upper end of the syringe reservoir, a compression spring MM5 is embedded therein, the upper end of which is supported against the upper end wall of the syringe reservoir and the lower end of which is acting on the end of syringe plunger MM4. After filling the syringe, the syringe plunger is locked by means of a locking lever MM6 with the spring MM5 being biased. After releasing the lock MM6, the syringe plunger MM4 is pressed downwards by the force of the compression spring MM5 and the sample contained in the syringe reservoir MM3 is injected into the furnace in a predetermined time interval or at a predetermined discharge rate.

This discharge of the predetermined sample amount at an exactly predetermined rate or in an exactly defined time interval is as important for reproducible results as the exact injection position and direction ensured by the particular design of the injection needle and the injection port.

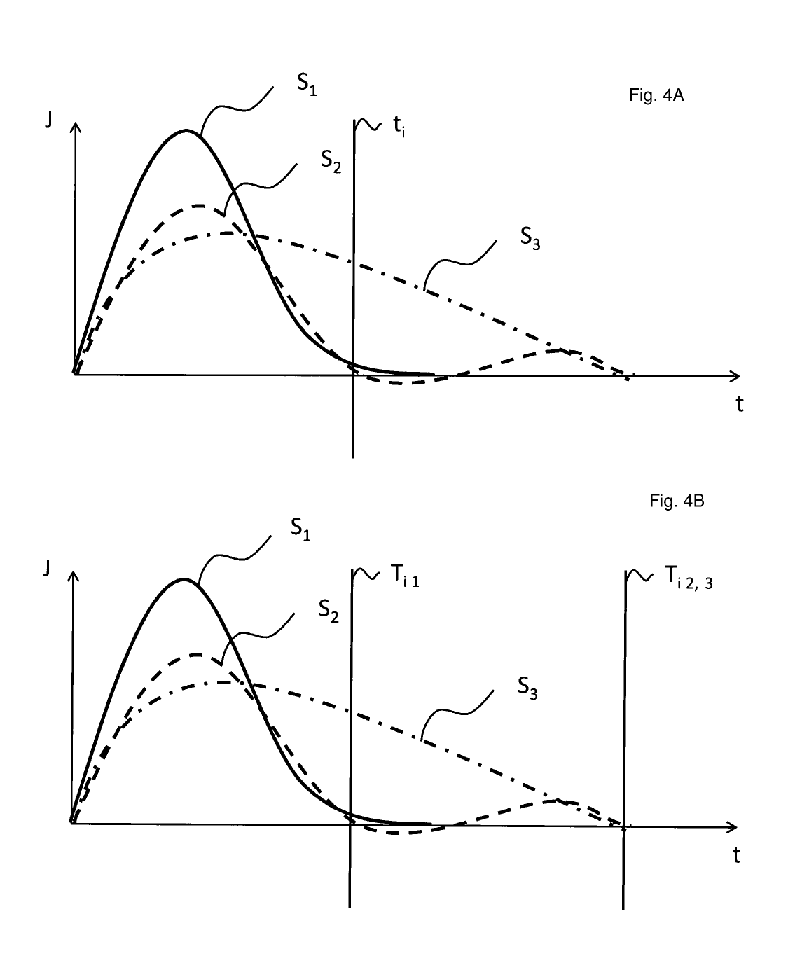

FIGS. 4A and 4B illustrate the effect of an adjustable integration time of the post-processing device A for processing an oxygen measurement signal detected as a function of time. Here, FIG. 4A shows three different curve shapes with fixed integration time tᵢ (according to the prior art). It can be seen that only the integration of the measurement signal S₁ shown by the solid line leads to a correct result, while the integration time set for the measurement signals S₂ and S₃ is obviously too short, with essential signal components not being detected. This is remedied by setting a longer integration time t_{i2, 3} for the signals S₂ and S₃, as shown in FIG. 4B, which can be performed by the operator of the analysis system at the post-processing device A depending on the detected signal curve shape.

However, the embodiment of the invention is not limited to this example, but a variety of modifications which are within the scope of ordinary skill in the art are possible.

1. An analysis system for analyzing water and wastewater, including an analysis device comprising
a device housing accommodating device components and having, on a housing surface, an inlet opening formed as an injection port for introducing a substance to be analyzed into a device component when said device housing is closed, and
a syringe having an outlet opening defined by an injection needle, a surface normal to the outlet opening coinciding with a longitudinal axis.

2. The analysis system according to claim 1, wherein said injection port is connected to a cylindrical reaction vessel for thermal disintegration of the substance to be analyzed, and includes a guiding element for guiding said syringe into a predetermined injection position.

3. The analysis system according to claim 2, wherein said guiding element is, in accordance with a shape of said injection needle, formed such that the longitudinal axis of the inserted injection needle coincides with the longitudinal axis of said reaction vessel, and comprises a cylindrical or conical guide sleeve.

4. The analysis system according to claim 3, wherein said guiding element comprises a stopper for limiting a depth of penetration of said injection needle into said reaction vessel.

5. The analysis system according to claim 11, wherein said ejection element is configured as a lockable compression spring, which acts on the plunger of said syringe.

6. An analysis system for analyzing water and waste water, according to claim 1, wherein a carrier gas supply is connected to said reaction vessel on an input side, said carrier gas supply including controls for setting a pressure and flow rate of a carrier gas to be supplied to said reaction vessel.

7. The analysis system according to claim 6, wherein said carrier gas supply comprises a first branch for supplying nitrogen and a second branch for supplying oxygen, wherein, in said first branch, a first pressure regulator and flow regulator for pressure regulation or flow regulation of supplied nitrogen are provided, and, in said second branch, a second pressure regulator and flow regulator for pressure regulation or flow regulation of oxygen to be mixed with the nitrogen are provided.

8. The analysis system according to claim 7, wherein said first and second pressure and flow regulators are configured and adapted such that a dilution by a factor of at least 10 is achieved during the mixing of the oxygen with the nitrogen.

9. An analysis system for analyzing water and waste water, according to claim 1, further comprising a detection and evaluation device for detecting a predetermined component of the substance to be analyzed and an adjustable post-processing device for manually controlled post-processing of a detection result.

10. The analysis system according to claim 9, wherein said detection device comprises at least one of an oxygen, a carbon dioxide, a nitrogen, or a phosphorus detector for detecting concentrations of at least one of oxygen, carbon dioxide, nitrogen, or phosphorus, respectively, in a time-dependent manner in a carrier gas flow exiting said reaction vessel, and said post-processing device for manually readjusting an integration time for integrating the respective concentration values detected in a time-dependent manner is provided.

11. The analysis system according to claim 1, wherein the syringe includes an automatically operating ejection element for ejecting a predetermined amount of substance in a predetermined injection period.
